(19) United States
(12) Patent Application Publication
(10) Pub. No.: US 2005/0176064 A1
(43) Pub. Date: Aug. 11, 2005

(54) Method for determining the number of receptors on a carrier

(76) Inventors: Holger Klapproth, Freiburg (DE); Ulrich Sieben, Reute (DE)

Correspondence Address: Patrick J O'Shea;O'Shea Getz & Kosakowski, Suite 912, 1500 Main Street, Springfield, VA, 01115, US

(21) Appl. No.: 10/520,039

(22) PCT Filed: Jun. 30, 2003
(86) PCT No.: PCT/EP03/06948

(30) Foreign Application Priority Data
Jul. 12, 2002 (DE) 102 31 684.8

(51) Int. Cl. G01N 33/53

(52) U.S. Cl. 435/7.1

(57) ABSTRACT

The invention relates to a method for determining the number of receptors on a carrier, in addition to a biosensor, especially a protein sensor, which can be produced using the method.

The present invention relates to a method for determining the number of receptors on a carrier and to a biosensor, especially a protein sensor, that can be produced using the method.

Biological systems depend on the interaction of biologically active macromolecules that bind other molecules, usually reversibly, through their three-dimensional surface structures and specific charge distributions. Besides reversible bonds, covalent bonds between molecules are also known that are utilized to bond molecules to surfaces by chemical methods. Molecules that have binding affinities for other molecules are collectively called receptors; they play a decisive role in the interaction and interplay of biological systems. Examples of receptors occurring in nature are enzymes, which catalyze the reaction of a given substrate; proteins, which enable the transport of charged molecules across a biomembrane; proteins modified by sugars (=glycoproteins), which permit contact with other cells; and antibodies, which circulate in the blood and recognize, bind, and inactivate constituents of pathogens such as bacteria and viruses. In the context of biologically active systems, DNA, the carrier of hereditary information, is also understood to be a receptor. DNA consists basically of two strands complementary to one another that form a double helix through base pairings and hydrogen bridges. Each individual DNA strand acts as a receptor in this context for its complementary DNA strand, which in turn assumes the function of a ligand.

All molecules that are specifically bound by a receptor are collectively called ligands; it is well known for many biologically active molecules that on the one hand they themselves bind other molecules, but on the other hand they are also bound by molecules. Therefore, depending on their particular binding partner, they are both ligands and receptors.

A number of test systems (assays) have been developed for studying interactions between receptors and ligands, by which the concentration of the ligand in a test solution can be determined qualitatively and/or quantitatively. Such test systems, because of the high specificity of receptor-ligand complexes, are used in criminology for examining crime suspects, in paternity tests, in cancer care, in prenatal diagnoses, in formulating family trees, in science and research, and for verifying successful vaccinations.

While the complete genomic DNA sequences of important model and research organisms such as bacteria (Bacillus subtilis, Escherichia coli) and yeasts (Saccharomyces cerevisiae) have been available in databases for years, the sequencing of the human genome has also now been completed in the course of the human genome project. Since the number of identified human genes is very much smaller than supposed, research on the function of the individual genes that are variably active in different tissues and organs has been gaining increasingly in importance.

Study of the protein portion of cells, which is also called proteome analysis, has become more and more important in the recent past, in addition to detailed research on DNA. Most pharmaceutically active substances that are used as drugs act through their effect on proteins. Such interactions cannot be analyzed, or can be analyzed only inadequately, by DNA analyses.

Clarification of differential gene expression is considered critical for understanding the development of many diseases. Therefore, many attempts have been made for many years to synthesize and classify artificially the largest possible number of biologically active molecules in the smallest space in order to be able to examine them with regard to their interaction with other molecules. Planar systems known as biochips or biosensors are used for the quantitative and qualitative detection of interaction partners or ligands in a sample to be analyzed, for example a sample of saliva or blood. The biosensors constitute a carrier on whose surface are developed a multitude of detection areas in a grid-like arrangement. In the production of such biosensors, the individual monomers were first applied by microdosing to the multitude of individual areas of the grid, on which a polymer is to be formed. This method is not suitable for broad screening studies, so that systems for light-controlled polymer synthesis using individual mask sets, familiar from the semiconductor industry, have been used to produce biosensors (Pease et al. (1994), PNAS, USA, Vol. 91, pp. 5022-5026).

The detection of a receptor-ligand complex formed on the surface of a biosensor plays the crucial role in the known test systems that are used for detecting ligands in samples to be analyzed. In ordinary systems, a calibration curve has to be prepared for calculating the concentration of the ligand, from which the number of ligand molecules or their concentration can be determined indirectly.

Systems are also used especially frequently in which it is attempted to label the ligands in the sample to be analyzed itself. It is a particular drawback to this, that the reaction of the ligand with a marker, for example a dye, can lead to a change in the configuration or conformation of the ligand and thus to a change in its surface structure. However, since precisely the three-dimensional surface is of critical importance for the binding of the ligand to the receptors fixed to the surface of the biosensor, the direct labeling of ligands as a rule does not provide satisfactory results.

Beyond this, molecular beacons have been developed that were described by Schonfield et al., (1997), Applied and Environmental Microbiology, Vol. 63, pp. 1143-1147, and by Tyagi and Kramer (1996), Nature Biotech., Vol. 14, pp. 303-308. Molecular beacons are DNA probes that have a short complementary sequence of nucleotides positioned at the 5′ and 3′ ends of the sample sequence, so that a stem-loop structure is formed in solution. A dye, particularly a fluorochrome, and a suitable quencher are placed at the ends of the stem-loop through linkers. This stem-loop structure constitutes the receptor in which the fluorochrome and the quencher are held close to one another through the stem-loop structure in the absence of a ligand, so that fluorescence is suppressed. However, when the single-strand loop interacts with a complementary target sequence (=ligand) and hybridizes in a stable manner, the stem-loop structure denatures. Because of this, fluorescence occurs since a more stable hybrid of loop and target sequence (=receptor-ligand complex) is developed that cannot coexist with the less stable internal base pairing of the stem hybrid. Since these probes fluoresce strongly only in the presence of a target sequence (a specific ligand), they can be used in solution without the need to remove unhybridized probe. Molecular beacons are highly specific, so that fluorescence is completely suppressed when the target sequence has a single incorrect base in the oligonucleotide chain. However, it is a drawback to the use of molecular beacons that they are limited to the detection of nucleic acids because of their mechanism of action. They cannot be used to detect other receptor-ligand complexes.

Up to now, biosensors have been used on which receptors have been immobilized to determine other receptor-ligand complexes, especially to detect antigen-antibody reactions. Thus far, it has been possible to determine the amount of bound receptor only inadequately. Determination of the amount usually depends on measuring how much fluid is released during the printing process of the sensor. The receptor density can also be determined statistically for individual printed sensors using various known staining techniques. It is felt to be a particular drawback here that at the time of measuring the interaction between receptor and ligand, no information can be obtained about the amount of immobilized receptor on the surface of the biosensor. Also, ordinary biosensors do not allow the receptor density to be measured on each individual sensor and at each measurement point.

Therefore, the task underlying this invention is to propose an improved method for determining the number of receptors on a carrier surface, with which the amount of actually immobilized receptor can be determined exactly. Furthermore, the detection of a formed receptor-ligand complex should occur specifically and should not be affected by the choice of marker.

This task is accomplished by a method for determining the number of receptors on a carrier in which the receptor-marker complexes are detected independently of the receptor-ligand complexes. In the method, a carrier is first prepared. At least one receptor is immobilized on the carrier, with the receptor being capable of interacting with a ligand and forming a specific receptor-ligand complex.

“Immobilizing” means any permanent connection of the receptor to the surface or the structure of the carrier. This interaction, for example, can depend on at least one covalent bond or at least one disulfide bridge. Furthermore, separable connections between receptor and carrier surface are conceivable and suitable, for which ionic interactions that can be detached simply by pH changes are advantageous. “Receptor-ligand complex” means any type of connection or interaction between receptor and ligand. The term “receptor-ligand complex” is thus not limited to the chemical definition of the term “complex.”

A signal molecule or a marker is then brought into contact with the receptor, whereby a receptor-marker complex is formed. The number of receptors on the carrier is then determined by detecting the receptor-marker complexes.

By detecting the receptor-marker complexes independently of the receptor-ligand complexes, the concentration of receptor can be determined directly. Since the binding constant, i.e. the affinity of the ligand for its receptor, is ordinarily known, the concentration of the ligand in a sample to be analyzed can be calculated from the receptor concentration and the binding constant. Furthermore, the production process for biosensors can be monitored using this method, because incorrectly printed or immobilized sensors can be readily recognized and left out.

Also, the receptor can be immobilized on the carrier and the marker can be brought into contact with the receptor at the same time in a single step. Thus, the method can be carried out especially easily and quickly, which is particularly important for routine diagnostic tests and so-called quick tests, which have to provide a reliable result in the shortest possible time.

The marker can also be brought into contact with the receptor first, to form the receptor-marker complex. These preformed receptor-marker complexes are then immobilized on the carrier through the receptor. Such a procedure is advantageous when the receptor-marker complex is particularly stable and is not impeded by subsequent binding of the receptor to the carrier surface.

In addition to the aforementioned process steps, the receptor can be brought into contact with a test sample that is to be examined for its content of ligand. The receptor can be incubated with the test sample after immobilizing the receptor on the carrier, or after the marker is brought into contact with the receptor, or after determining the number of receptors on the carrier.

If the receptor is brought into contact with a test sample that is to be tested for its content of ligand, it is advantageous to detect the formed receptor-ligand complexes directly and independently of the formed receptor-marker complexes.

The carrier can be a semiconductor. Its surface can consist of silicon or of semimetal oxides. SiOₓ or aluminum oxide are especially advantageous.

The receptor used in the context of the invention can be any molecule with binding affinity for a given ligand. Receptors can be naturally occurring or can be produced synthetically. They can also be in their natural state or as aggregates with other molecules. Receptors can bind covalently or non-covalently to the ligands, directly or indirectly through specific binding substances or binding molecules. Examples of receptors are enzymes, antibodies, particularly monoclonal or polyclonal antibodies, as well as functional fragments thereof, antisera, proteins, oligo- and polypeptides, cell membrane receptors, nucleic acids, especially DNA, RNA, cDNA, PNA, oligo- and polynucleotides, sugar constituents such as saccharides, especially mono-, di-, tri-, oligo-, and polysaccharides, as well as lecithin, cofactors, cellular membranes, organelles, and lipids and their derivatives.

It is essential for receptors to form a receptor-ligand complex with the corresponding ligands by their molecular recognition. Consequently, ligands are molecules that are recognized by a given receptor. They can also occur naturally or can be produced synthetically. Examples of known ligands are agonists and antagonists of cellular membrane receptors, toxins, viral and bacterial epitopes, especially antigens, hormones (opiates, steroids, etc.), peptides, enzymes, enzyme substrates, and cofactors.

Although the binding between receptor and ligand in the receptor-ligand complex is highly specific, it can nevertheless be separable, for example by changing the temperature, pH, ionic concentration, or salt content of the surrounding medium, or by the presence of competing molecules.

If the fluorochrome of the ligand has a longer fluorescence lifetime than the fluorochrome of the marker, the markers may differ highly selectively from one another. A similar effect can be produced by using dyes with differing excitation and emission spectra. If the ligand and the marker bind at the same position on the receptor and thus compete with one another for this binding (so-called competitive antagonism), it is advantageous for the marker to have less binding affinity to the receptor.

The binding between receptor and marker in the receptor-marker complex can be made separable, so that the marker can be displaced from its binding to the receptor by suitable competitive substances, and can be replaced by other markers.

The receptors are labeled with markers statistically, i.e. not every single receptor has to be labeled individually. Nevertheless, on average there are n markers on n receptors. The labeling can also amount to a multiple of n. It is essential for the markers not to interfere with the principle of measurement.

The markers can have reactive groups; chemically reactive groups with high specificity, for example such as thiol groups, are especially suitable as reactive groups. The binding properties of the ligand to the receptor are not impaired to any great extent by such chemically reactive groups.

The marker can be a dye, in particular a luminescent dye, and especially a chemoluminescent, photoluminescent, or bioluminescent dye.

If the marker is a fluorescent dye, it may then have a fluorochrome. Rhodamine, especially tetramethylrhodamine isothiocyanate (=TRITC) is particularly suitable in this case. Such fluorochromes can be used as maleimides for conjugation. If the receptor is an antibody, it can be conjugated with reactive dyes. A number of examples of this can be found in the publication “Bioconjugate Techniques” by G. T. Herrmannson, Academic Press 1996.

So-called chimerical proteins that are assembled synthetically from protein constituents of differing origins, for example biological and synthetic origins, can also be used. Thus, for example, an antibody in which the constant region (Fab region) has been replaced by a fluorescing protein so that only the variable regions are retained for antigen recognition, can be used as a receptor. In particular, the fluorescing protein can be a green fluorescent protein (GFP) or a blue fluorescent protein (BFP).

The receptor can also have inherent fluorescence. Such inherent fluorescence is known in particular for the naturally occurring amino acid tryptophan, which occurs in almost all proteins of any size. Accordingly, when the receptor is an antibody, protein, or oligopeptide, in which at least one tryptophan appears, the inherent fluorescence of this amino acid can be used for detection.

The binding between receptor and marker in the receptor-marker complex has a fluorescence half-life in the nanosecond range.

The receptor-marker complex may show a fluorescence resonance energy transfer (=FRET). In this system, there is energy transfer between a donor, which gives up the energy, and an acceptor, which accepts the energy.

The fluorescence of the FRET can be modified by the interaction of the ligand with the receptor. The donor and the acceptor of the FRET can be immobilized on the receptor. The donor and acceptor are separated spatially from one another by binding the ligand to the receptor, so that fluorescence is quenched by the acceptor, with the acceptor being a fluorochrome. On the other hand fluorescence can be created by the donor, with the acceptor being called a fluorescence quencher. The ligand itself can also act as a donor, so that it either fluoresces or quenches. It is also conceivable for the ligand to bring the FRET donor and acceptor in immediate direct contact with one another by its binding to the receptor to form the receptor-ligand complex.

FRET can also function as acceptor with both a quencher and a fluorochrome. If the acceptor is a quencher, the fluorescence is quenched. If the acceptor is a fluorochrome, the energy of the donor is again emitted by the acceptor as fluorescent light.

In the same way, ligands that are themselves fluorescence-labeled can be used. In this way, a competitive assay is possible using a likewise labeled competitor, which can be a fluorescence-labeled ligand, for example. The competitor produces (or extinguishes) a fluorescent signal on the receptor. It is especially advantageous here to produce a signal, because non-specific binding of the ligand/competitor in regions outside the receptor is not signal-forming.

The marker can assume any desired detectable form; the marker in particular can be a microparticle.

This invention also relates to a biosensor, especially a protein sensor, that can be produced according to the method according to the invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. Method for determining the number of receptors on a carrier, comprising:
(a) preparing a carrier;
(b) immobilizing at least one receptor on the carrier, with the receptor having the ability to interact with a ligand and to form a receptor-ligand complex;
(c) after immobilization of at the at least one receptor on the carrier, bringing a marker in contact with the receptor, in order to form a receptor-marker complex with separable binding between receptor and marker; and
(d) determining the number of receptors on the carrier by detecting the receptor-marker complexes;
wherein the receptor-marker complexes are detected independently of receptor-ligand complexes.

28. The method of claim 27, comprising:
(i) bringing the receptor in contact with a test sample that is to be examined for its content of ligands.

29. The method of claim 28, comprising:
(ii) following step (i), detecting the receptor-ligand complexes.

30. The method of claim 27, wherein the carrier is a semiconductor with a surface of silicon, semimetal oxides, especially SiOₓ, or aluminum oxide.

31. The method of claim 27, wherein the receptor is selected from the group consisting of antibodies, especially monoclonal or polyclonal antibodies, and functional fragments thereof; proteins, oligo- and polypeptides, nucleic acids, especially DNA, RNA, cDNA, PNA, oligo- and polynucleotides; as well as saccharides, especially mono-, di-, tri-, oligo-, and polysaccharides.

32. The method of claim 27, wherein the binding between receptor and ligand in the receptor-ligand complex is separable.

33. The method of claim 27, wherein the binding between receptor and ligand has a half-life in the range of at least microseconds.

34. The method of claim 27, wherein n markers or a multiple of n markers are associated with n receptors.

35. The method of claim 27, wherein the marker has reactive groups, especially thiol groups.

36. The method of claim 27, wherein the marker comprises a luminescent dye, a chemoluminescent, a photoluminescent dye, or a bioluminescent dye.

37. The method of claim 27, wherein the marker comprises a fluorescent dye, preferably a fluorochrome, and with greater preference a rhodamine, especially tetramethylrhodamine isothiocyanate.

38. The method of claim 27, wherein the receptor comprises inherent fluorescence.

39. The method of claim 38, wherein the amino acid tryptophan provides the inherent fluorescence.

40. The method of claim 38, wherein the binding between receptor and marker has a fluorescence half-life in the range of nanoseconds.

41. The method of claim 27, wherein the receptor-marker complex includes fluorescence resonance energy transfer.

42. The method of claim 41, wherein the fluorescence of the fluorescence resonance energy transfer is modified by the interaction of the ligand with the receptor.

43. The method of claim 41, wherein the receptor has the donor and the acceptor of the fluorescence resonance energy transfer.

44. The method of claim 41, wherein the fluorescence is produced by the donor or the fluorescence is quenched by the acceptor.

45. The method of claim 41, wherein the ligand acts as the donor of the fluorescence resonance energy transfer.

46. The method of claim 41, wherein the ligand brings the donor and the acceptor of the fluorescence resonance energy transfer directly into contact.

47. The method of claim 41, wherein fluorescence-labeled ligands are used.

48. The method of claim 42, wherein the marker is a microparticle.

49. A method of determining the number of receptors using a biosensor, comprising:
(a) preparing a semiconductor carrier;
(b) immobilizing at least one receptor on the carrier, with the receptor having the ability to interact with a ligand and to form a receptor-ligand complex;
(c) after immobilization of at the at least one receptor on the carrier, bringing a marker in contact with the receptor, in order to form a receptor-marker complex with separable binding between receptor and marker; and
(d) determining the number of receptors on the carrier by detecting the receptor-marker complexes;
wherein the receptor-marker complexes are detected independently of receptor-ligand complexes, the marker comprises a luminescent dye, a chemoluminescent, a photoluminescent dye, or a bioluminescent dye.
